(19) United States
(12) Patent Application Publication
(10) Pub. No.: US 2024/0197932 A1
(43) Pub. Date: Jun. 20, 2024

(54) Biodegradable Alginate Microspheres

(71) Applicant: Plus Therapeutics, Inc., Austin, TX (US)

(72) Inventors: Cheryl D. Rice, San Diego, CA (US); Daniel O. Ortega, San Antonio, TX (US); Marc Hedrick, Charlottesville, VA (US)

(73) Assignee: Plus Therapeutics, Inc., Austin, TX (US)

(21) Appl. No.: 18/390,264

(22) Filed: Dec. 20, 2023

Related U.S. Application Data
(60) Provisional application No. 63/433,889, filed on Dec. 20, 2022.

(51) Int. Cl. A61K 51/12; A61K 9/127; A61K 51/06

(52) U.S. Cl. CPC A61K 51/1237; A61K 9/1277; A61K 51/06

(57) ABSTRACT

Disclosed herein is a method of preparation and composition of biodegradable alginate microspheres that in an embodiment, are used in treating at least one of the following diseases hepatocellular carcinoma, colorectal liver metastases, paraganglioma liver metastases, n neuroendocrine liver metastases, gastrointestinal liver metastases, breast liver metastases, melanoma liver metastases, pancreatic liver metastases, cholangiocarcinoma liver metastases, colorectal lung metastases, renal lung metastases, cirrhosis-associated thrombocytopenia, metastatic extrahepatic bile duct cancer, glioblastoma, renal cell carcinoma, prostate cancer, and uterine fibroids. The method of preparation of the biodegradable alginate microspheres can, in some embodiments, utilize a microfluidic cassette.

### CROSS-REFERENCE TO RELATED APPLICATIONS

This application claims the benefit of U.S. Provisional Application No. 63/433,889, filed Dec. 20, 2023.

### TECHNICAL FIELD

This disclosure is generally related to the production of microspheres; more specifically to the production of loaded alginate microspheres.

### BACKGROUND OF THE INVENTION

Alginate microspheres are popular for use in drug delivery. Alginate is an attractive choice because it is biocompatible and biodegradable. Alginate microspheres can be used to protect the encapsulated drug from conditions within the body.

Microparticles, such as alginate microspheres, produced by standard production methods frequently have a wide particle size distribution, lack uniformity, fail to provide adequate release kinetics or other properties, and are difficult and expensive to produce. In addition, the microparticles may be large and tend to form aggregates, requiring a size selection process to remove particles considered to be too large for administration to patients by injection or inhalation. This requires sieving and results in product loss.

It would be advantageous to have a process of producing biodegradable alginate microspheres that provides a predictable particle size distribution and uniformity along with ease of production.

### SUMMARY OF THE INVENTION

An embodiment of the disclosure is a method of manufacturing a polymeric matrix encapsulating liposomes comprising adding at least one liposome to a first channel on a microfluidic cassette; adding at least one cross-linkable polymer to a second channel on the microfluidic cassette; combining the at least one liposome and at least one crosslinkable polymer to form a solution; pushing the solution through a third channel on the microfluidic cassette to shear the solution to create individual spheres; and adding an aqueous solution comprising a cross-linker to a fourth channel on the microfluidic cassette; wherein the cross-linker and cross-linkable polymer form the polymeric matrix encapsulating liposomes. In an embodiment, the at least one cross-linkable polymer is alginate. In an embodiment, the alginate is composed of mannuronate and guluronate stereomonomers. In an embodiment, the alginate is composed of a mannuronate to guluronate ratio of less than or equal to 1. In an embodiment, the alginate is composed of a mannuronate to guluronate ratio of greater than or equal to 1.5. In an embodiment, the cross-linker is a divalent cation. In an embodiment, the cross-linker is a calcium salt. In an embodiment, the cross-linker is a barium salt. In an embodiment, the method further comprises washing the polymeric matrix encapsulating liposomes to remove excess cross-linker. In an embodiment, the at least one liposome is loaded with at least one active agent before adding the liposome to the first channel on the microcassette. In an embodiment, the at least one active agent is radioactive. In an embodiment, the at least one active agent is a beta emitting radioactive nucleotide chelate. In an embodiment, the beta emitting radioactive nucleotide chelate comprises Re188. In an embodiment, the beta emitting radioactive nucleotide chelate comprises BMEDA. In an embodiment, the diameter of the first channel, the second channel, the third channel, and the fourth channel is 20-120 μm respectively. In an embodiment, the method further comprises an aspect ratio of the microspheres of 0.7-1.3 μm. In an embodiment, the method further comprises a difference in flow rate between a dispersed phase and a continuous phase of 0-100 μL/min. In an embodiment, the method further comprises a stream temperature of 5° C.-55° C. In an embodiment, the method further comprises a stream pressure of 0-1500 mbar.

An embodiment of the disclosure is a composition manufactured by the method of claim 1 comprising at least one liposome; at least one active agent encapsulated in an intraliposomal aqueous compartment of the liposome; a cross-linked polymeric matrix, wherein the polymeric matrix is water-insoluble and water-absorbed; wherein the at least one liposome has a diameter between about 1 nm-200 nm; and wherein the at least one liposome is embedded in the cross-linked polymeric matrix. In an embodiment, the cross-linked polymeric matrix comprises at least one active agent in free form. In an embodiment, the composition further comprises an aqueous medium surrounding the cross-linked polymeric matrix embedded with the at least one liposome. In an embodiment, the aqueous medium comprises at least one active agent in free form. In an embodiment, the at least one active agent is radioactive. In an embodiment, the at least one radioactive agent is a beta emitting nucleotide chelate. In an embodiment, the cross-linked polymeric matrix comprises alginic acid. In an embodiment, the at least one liposome comprises at least one ionic salt inside the intraliposomal aqueous compartment to create an ion gradient. In an embodiment, the at least one liposome comprises at least one buffering agent inside the intraliposomal aqueous compartment to create a pH gradient. In an embodiment, the at least one liposome comprises at least one radio-opaque material. In an embodiment, the cross-linked polymeric matrix has a diameter of about 10-90 μm. In an embodiment, the cross-linked polymer is alginate.

An embodiment of the disclosure is a method of using the polymeric matrix encapsulating liposomes manufactured using the method, wherein the active agent loaded in the polymeric matrix actively destroys tumor cells.

An embodiment of the disclosure is a method of using the polymeric matrix encapsulating liposomes manufactured using the method to treat at least one of the following diseases using radioembolization: hepatocellular carcinoma, colorectal liver metastases, paraganglioma liver metastases, n neuroendocrine liver metastases, gastrointestinal liver metastases, breast liver metastases, melanoma liver metastases, pancreatic liver metastases, cholangiocarcinoma liver metastases, colorectal lung metastases, renal lung metastases, cirrhosis-associated thrombocytopenia, metastatic extrahepatic bile duct cancer, glioblastoma, renal cell carcinoma, prostate cancer, and uterine fibroids.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The present technology will be better understood on reading the following detailed descriptions of non-limiting embodiments thereof, and on examining the accompanying drawings, in which:

FIG. 1 depicts a flow chart of the preparation of the microspheres;

FIG. 2 depicts an embodiment of a microfluidic cassette.

FIG. 3A, FIG. 3B, FIG. 3C, and FIG. 3D depict particle size data comparing 188RNL BAM Intermediates (PTN12-066), KI Liposomes (PTN13-008), BAM KI Liposomes 1:1 (PTN13-014), and BAM KI Liposomes 2:1 (PTN13-015); and

FIG. 4A and FIG. 4B depict X ray fluoroscopy images of the radiopaque prototype, 188RNL-BAM-KI (4A) and the non-radiopaque prototype, 188RNL-BAM (4B). The settings were 43 kV, 3.7 mA, 84.55 mGy, for 4 minutes.

### DETAILED DESCRIPTION

Unless defined otherwise, all technical and scientific terms used herein have the same meaning as is commonly understood by one of skill in the art to which this disclosure belongs.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment,” “an embodiment,” “certain embodiments,” or “other embodiments” of the present invention are not intended to be interpreted as excluding the existence of additional embodiments that also incorporate the recited features. Furthermore, reference to terms such as “above,” “below,” “upper,” “lower,” “side,” “front,” “back,” or other terms regarding orientation are made with reference to the illustrated embodiments and are not intended to be limiting or exclude other orientations.

Disclosed herein are liposome-alginate sphere compositions and a novel processing method to manufacture the spheres using microfluidic chips, cartridges, or cassettes. In an embodiment, the microfluidic cassette is made of at least one of polydimethylsiloxane (PDMS), glass, silicon, polymethylmethacrylate (PMMA), polycarbonate, and cyclic olefins. In an embodiment, the diameter of the microfluidic channels ranges from 1 μm to 1000 μm. In an embodiment, the size of the microfluidic chip ranges from one-half inch to 4 inches or about 1 centimeters to 10 centimeters.

Certain embodiments are directed to compositions comprising and a method for producing liposome-containing alginate microspheres. In an embodiment, the liposomes encapsulate one or more substances. Substances that can be encapsulated in liposomes and loaded into alginate microspheres include radiotherapeutics (including but not limited to rhenium-188), radio labels (including but not limited to technetium-99m), chemotherapeutics (doxorubicin), magnetic particles (including but not limited to 10 μm iron nanoparticles), and radio-opaque material (including but not limited to iodine contrast).

In an embodiment, the imaging agent is a radiolabel selected from the group comprising a radioisotopic element selected from the group consisting: of astatine, bismuth, carbon, copper, fluorine, gallium, indium, iodine, lutetium, nitrogen, oxygen, phosphorous, rhenium, rubidium, samarium, technetium, thallium, yttrium, and zirconium.

In an embodiment, the radiolabel is selected from the group comprising zirconium-89 (⁸⁹Zr), iodine-124 (¹²⁴I), iodine-131 (¹³¹I), iodine-125 (¹²⁵I) iodine-123 (¹²³I), bismuth-212 (²¹²Bi), bismuth-213 (²¹³Bi), astatine-211 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵³Sm), lutetium-177 (¹⁷⁷Lu), technetium-99m (^{99m}Tc), gallium-67 (⁶⁷Ga), indium-111 (¹¹¹In), thallium-201 (²⁰¹Tl), carbon-11 (¹¹C), nitrogen-13 (¹³N), oxygen-15 (¹⁵O), fluorine-18 (¹⁸F), and rubidium-82 (⁸²Ru).

In certain aspects, rhenium-188 (¹⁸⁸Re) liposomes in alginate microspheres (Rhe-LAMs) can be used for treatment of liver tumors, specifically hepatocellular carcinoma (HCC). In an embodiment, HCC treatment can be through radioembolization, where the microspheres block the blood supply to the tumor from the artery, while the rhenium-188 also delivers a high dose of radiation that is primarily targeted to the cancer cells.

In an embodiment, microfluidic channels are used to produce alginate microspheres-containing liposomes (liposome embedded polymeric matrices). A microfluidic cassette mixes two fluid streams, one stream comprising liposomes and one stream comprising alginate, with the two fluid streams crossing in a microfluidic channel. The mixed solution is then pushed continuously through another microfluidics channel where the solution is sheared to create spheres. Spheres will have a mean diameter of 10-90 μm. Then, the spheres interact with a of solution containing a divalent ion such as calcium or barium, where the spheres are gelated with liposomes entrapped in the alginate matrix. In one embodiment the solution is composed of 1% to 50% calcium salt or barium salt. In a more preferred embodiment, the solution is composed of 1% to 40% calcium salt or barium salt. In a further preferred embodiment, the solution is composed of 20% to 30% calcium salt or barium salt. Characteristics of the alginate-liposome microspheres are determined in this process by variables such as the angle between interacting channels, channel diameter, stream flow rate, stream temperature, stream pressure, and the ratio of mannuronate to guluronate in the alginate. The microfluidic method yields alginate microspheres (containing liposomes) with a narrow particle size distribution and of uniform shape.

The effect of particle shape on flow dynamics can be significant. In a study using direct numerical simulation (DNS), it was observed that the local accumulation of spheres near the wall decreases for spheroids with increasing aspect ratio (7). This means that the distribution of the particles in the flow is affected by their shape. Additionally, spheroidal particles were found to rotate slower near the wall and tend to align their major axes with the flow streamwise direction (8). This indicates that the orientation of the particles in the flow is also affected by their shape. Furthermore, despite the lower rotation rates, a higher intermittency in the rotational rates was observed for spheroids, and this increased with the aspect ratio. This suggests that the motion of the particles is affected by their shape, and that spheroidal particles with a higher aspect ratio exhibit more erratic behavior (7). Overall, these results demonstrate that particle shape plays a significant role in modulating the flow dynamics.

The flow dynamics of a sphere and a droplet can be quite different (7). For example, a sphere will typically move in a more predictable and symmetrical manner compared to a droplet, which may exhibit more irregular and unpredictable motion (7). Additionally, the presence of surface tension in a droplet can cause it to deform and change shape as it moves, which can affect its trajectory and interaction with the surrounding fluid. Overall, the differences in shape between a sphere and a droplet can lead to distinct flow dynamics and behavior in a fluid (8,9).

Aspect ratio is the ratio of the longest axis of an object to its shortest axis. In the case of a particle that can be a perfect sphere to slightly elongated, the aspect ratio would vary depending on the degree of elongation. For a perfect sphere, the longest and shortest axes are the same, so the aspect ratio would be 1. As the particle becomes slightly elongated, the aspect ratio would increase.

In an embodiment the aspect ratio of the microspheres is 0.7 to 1.3. In a preferred embodiment, the aspect ratio for the microsphere is 0.8 to 1.2. In a further preferred embodiment, the aspect ratio for the microsphere is 0.9 to 1.1.

In an embodiment, the channel diameter is 20-120 μm. In a preferred embodiment, the channel diameter is 30-100 μm. In a more preferred embodiment, the channel diameter is 40 -80 μm. In a further preferred embodiment, the channel diameter is 60-80 μm. In an embodiment, the channel diameter of the first channel, second channel, third channel, and fourth channel are between 20-120 μm respectively. In an embodiment, any of the channels can have a diameter that is between 20-120 μm. The diameter may or may not be the same as that of any of the other channels.

In an embodiment, the difference in flow rate between the dispersed phase and the continuous phase is 0 to 100 μL/min. In a preferred embodiment, the difference in flow rate between the dispersed phase and the continuous phase is 0-30 μL/min. In a more preferred embodiment, the difference in flow rate between the dispersed phase and the continuous phase is 0-15 μL/min. In a further preferred embodiment, the difference in flow rate between the dispersed phase and the continuous phase is 5-10 μL/min.

In an embodiment, the stream temperature is 5° C. to 55° C. In a preferred embodiment, the stream temperature is 15° C. to 40° C. In a more preferred embodiment, the stream temperature is 10° C. to 40° C. In a further preferred embodiment, the stream temperature is 20° C. to 30° C.

In an embodiment, the stream pressure is 0 to 1500 mbar. In a preferred embodiment, the stream pressure is 50 to 100 mbar. In a more preferred embodiment, the stream pressure is 300 to 800 mbar. In a further preferred embodiment, the stream pressure is 600 to 700 mbar.

A pH gradient between the fluid and the aqueous core of the liposome is required to encapsulate an active pharmaceutical ingredient. In an embodiment, the fluid is continuous. Standard production methods frequently use a washing procedure in which the calcium chloride solution is removed by creating a pellet of alginate microspheres using centrifugation. The supernatant is discarded and re-suspended with a suitable ionic buffer. This process is repeated until acceptable levels of calcium chloride are achieved.

In an embodiment, tangential flow filtration (TFF) can be used to remove calcium chloride and for buffer exchange. TFF is a separation process in which the fluid is passed parallel to the filter instead of perpendicularly through the membrane. TFF can be used to concentrate a solution for diafiltration, removing salts. In an embodiment, the TFF membrane is a hydrophilic membrane. In an embodiment, the TFF membrane is polyethersulfone (PES). In an embodiment, Minimate™ Tangential Flow Filtration Capsules with Omega™ Membrane can be used to remove calcium chloride.

Certain embodiments described herein include the pre-encapsulation of a water-soluble radio-opaque material into the pH gradient liposomes. Water soluble materials can be incorporated into the liposomes via passive encapsulation. Liposomes form spontaneously when dissolved liposome components are exposed to aqueous solutions. As liposomes form, the aqueous solution is entrapped in the lumen of the liposome. In one embodiment, water soluble potassium iodide is encapsulated into the pH gradient liposome. In an embodiment, ammonium sulfate liposomes can be prepared as in U.S. Pat. Publ. No. 20220249374 (10).

An embodiment of the disclosure is a composition of matter comprising liposomes encapsulating in their intraliposomal aqueous compartment at least one active agent, the liposomes having a diameter of at most 200 nm and being embedded in a water insoluble, water absorbed cross-linked polymeric matrix. In an embodiment, the polymeric matrix comprises an amount of at least one active agent in free form. In an embodiment, an aqueous medium is added surrounding the polymeric matrices embedded with liposomes. In an embodiment, the aqueous medium comprises an amount of at least one active agent in free form. In an embodiment, at least one active agent is radioactive in some capacity. In an embodiment, the cross-linked polymeric matrix is alginic acid. In an embodiment, the liposomes contain at least one ionic salt inside the intraliposomal aqueous compartment to create an ion gradient. In an embodiment, the liposomes contain at least one buffer agent inside the intraliposomal aqueous compartment to create a pH gradient. In an embodiment, the liposomes contain at least one radio-opaque material. In an embodiment, the at least one radioactive agent is a beta emitting nucleotide chelate.

Polysaccharides, such as alginates, are widely used in various fields due to their structural properties, biocompatibility, and safety. When alginate is combined with divalent cations, a hydrogel is formed that has been shown to be biocompatible and capable of encapsulating cells (1). However, previous research has demonstrated that the purity of alginate can affect its biocompatibility in implants (2). Alginate is a copolymer composed of mannuronate (M) and guluronate (G) stereomonomers (3, 4). The affinity of divalent cations, such as calcium, is stronger for G isomers due to their atomic arrangement (4, 5). Two G monomers can form an “egg-box” structure in which a calcium ion sits and crosslinks two polymer chains (4). As a result, the M:G ratio can affect the crosslinking capacity and physical properties of a calcium alginate gel (6). Higher proportions of mannuronic acid can produce softer gels, while higher proportions of guluronic acid can produce stiffer gels with tighter pores. This property has been used in the production of biodegradable implants, where different ratios of mannuronic and guluronic acid can be used to achieve the desired rigidity of the gel matrix.

In an embodiment, the liposome embedded polymeric matrices have a mean diameter below 80 μm. In an embodiment, the cross-linked polymer is alginate. In an embodiment, the alginate is composed of a mannuronate to guluronate ratio of less than or equal to 1. In an embodiment, the alginate is composed of mannuronate to guluronate ratio of greater than or equal to 1.5.

An embodiment of the disclosure is a method comprising mixing (i) liposomes, (ii) at least one cross-linkable polymer , and (iii) an aqueous solution comprising a cross-linker such that the cross-linker and cross-linkable polymer form a water insoluble , water absorbed cross-linked polymer in which the liposomes are embedded therein. In an embodiment, the composition is washed to remove excess amount of the cross-linker. In an embodiment, microfluidic channels are used to mix the liposomes, aqueous cross linker solution, and cross linkable polymer. An embodiment of the disclosure is a method to shear the cross-linked polymer into individual particles comprising a microfluidic channel. In an embodiment, the liposomes within the microsphere are loaded with at least one active agent before mixing. In an embodiment, the at least one active agent is radioactive in some capacity. In an embodiment, the at least one radioactive agent is a beta emitting nucleotide chelate. In an embodiment, the at least one of the chelating agents is BMEDA. In an embodiment, the at least one of the chelating agents is DOTA.

An embodiment of this disclosure is a method of loading at least one chelated agent into liposome polymeric matrices.

An embodiment of the disclosure is a method to use the composition herein to treat a disease wherein liposome embedded polymeric matrices embolize blood vessels within unresectable tumors. In an embodiment, blood vessels within unresectable tumors are embolized using at least one material that is known as biodegradable. In an embodiment, the active agent loaded in the polymeric matrices actively destroys tumor cells.

An embodiment of the disclosure is a method to treat at least one of the following diseases using radioembolization: (i) Hepatocellular carcinoma; (ii) Colorectal liver metastases; (iii) Paraganglioma liver metastases; (iv) Neuroendocrine liver metastases; (v) Gastrointestinal liver metastases; (vi) Breast liver metastases; (vii) Melanoma liver metastases; (viii) Pancreatic liver metastases; (ix) Cholangiocarcinoma liver metastases; (x) Colorectal lung metastases; (xi) Renal lung metastases; (xii) Cirrhosis-associated thrombocytopenia; (xiii) Metastatic extrahepatic bile duct cancer; (xiv) Glioblastoma; (xv) Renal cell carcinoma; (xvi) Prostate cancer; and (xvii) Uterine fibroids.

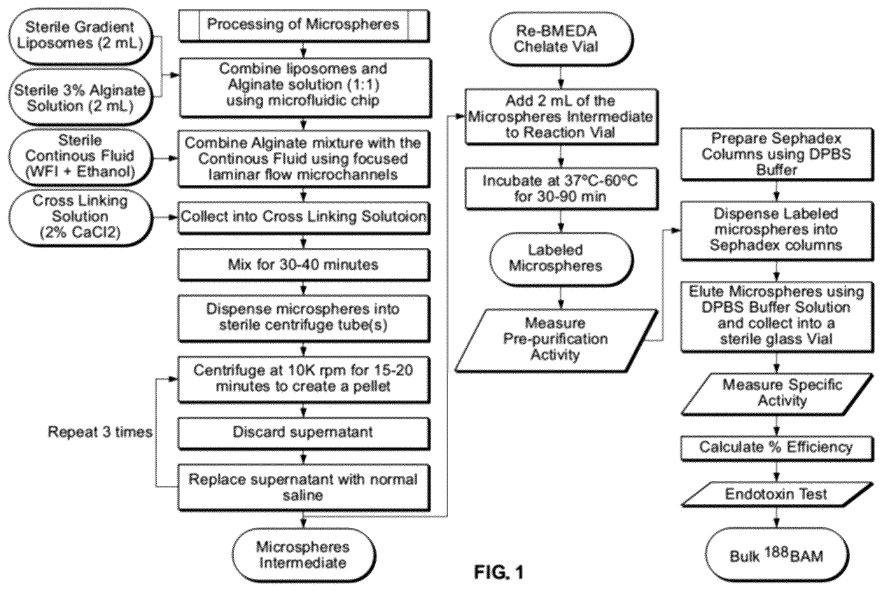

To process the microspheres, 2 ml of sterile gradient liposomes are combined with 2 ml of a 3% sterile alginate solution to form a 1:1 alginate mixture. FIG. 1. The mixture is mixed until uniform. The alginate mixture is combined with the sterile continuous fluid of water for injection (WFI) and ethanol using focused laminar flow microchannels. This is collected into a 2% CaCl₂ crosslinking solution and mixed for 10-90 minutes before dispensing the microspheres into sterile centrifuge tube(s). The tubes are centrifuged at 10K rpm for 15-20 minutes to create a pellet, the supernatant is discarded, and normal saline is added to the tube with the pellet. The centrifugation, supernatant removal, and addition of saline is repeated 3 times resulting in the microsphere intermediate.

Two ml of the microsphere intermediate is added to a vial of Re-BMEDA chelate and is incubated at 37° C. for 60 minutes, resulting in labeled microspheres.

The pre-purification activity of the labeled microspheres is measured. Sephadex columns are prepared using Dulbecco's Phosphate-Buffered Saline (DPBS). The labeled liposomes are dispensed into the Sephadex columns. The labeled liposomes are eluted using DPBS Buffer and collected into a sterile glass vial. The specific activity is measure and percent efficiency calculated. A test is performed to make sure that there is no endotoxin contamination.

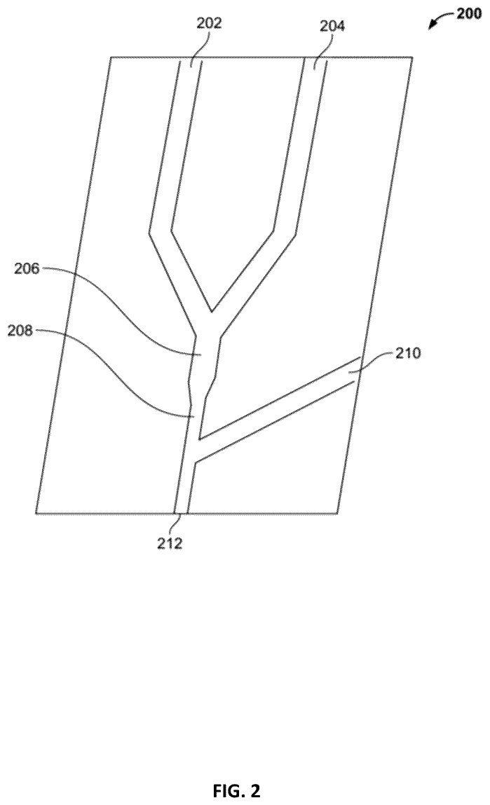

A method of making alginate microspheres involves mixing 3% Alginic acid solution and Ammonium Sulfate Liposomes into a solution using a microfluidic device, 200, that provides continuously focused laminar flow. In an embodiment, the microfluidic device will be comprised of at least one central fluid inlet channel, 202 and 204, and at least one outer fluid inlet channel, 212. FIG. 2. The main principle of microfluidic mixers is to decrease diffusion length and diffusion time between different fluid components. In an embodiment, the microfluidic devise will be designed to provide either chaotic micro-mixing or microfluidic hydrodynamic focusing.

In an embodiment, the solution is mixed by having two streams, one in inlet channel 202 with liposomes, and the other in inlet channel 204 with alginate, cross in a microfluidic channel 206. The mixed solution is then pushed continuously through another microfluidics channel, 208, where the solution is sheared to create spheres. Spheres will have a mean diameter of 10-80 μm. Then, the spheres interact with a stream of 30% calcium chloride solution from channel 210, where the spheres are gelated with liposomes entrapped in the alginate matrix. Characteristics of the alginate-liposome microspheres are determined in this process by variables such as angle between interacting channels, channel diameter, stream flow rate, stream temperature, and stream pressure. The microfluidics will take place in a microfluidic cassette or chip, with process variables being controlled by a relevant controller. In an embodiment, The range of the angles between the interacting channels is from ϑ=1° to ϑ0=85°.

The alginate-liposome microspheres are then loaded with 188Re-BMEDA (Rhenium-188 BMEDA) by incubating the microspheres and 188Re-BMEDA at an elevated temperature for an hour. The BMEDA solution is created by interacting Rhenium-188 generated from Tungsten-188 with a BMEDA and Sodium Glucoheptonate solution at 80° C. for one hour. The 188Re-BMEDA is encapsulated within the Ammonium Sulfate Liposomes in the alginate microspheres.

### EXAMPLES

### Example 1

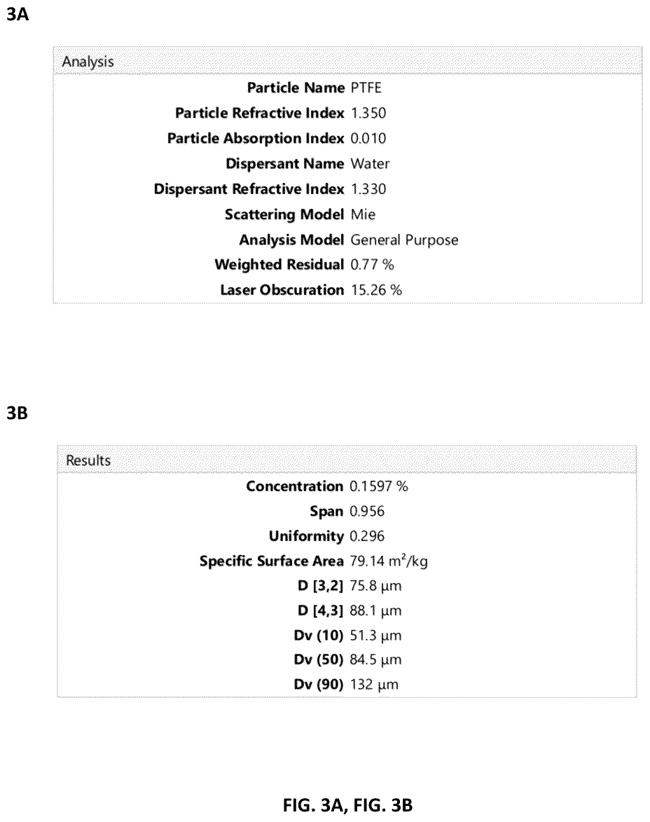

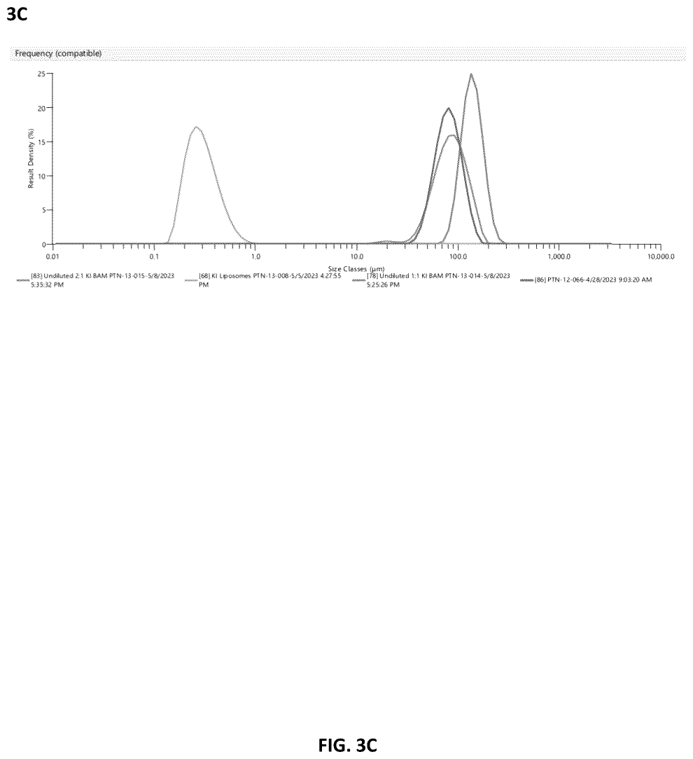

188RNL-BAM intermediates were manufactured using the atomizer nozzle method. The intermediates can be used for including, but not limited to, characterization and pre-clinical studies (FIG. 3C, PTN12-066).

Materials used included DPBS, 3% alginic acid, liposomes, and 20 gL CaCl2.

The nozzle and tubing were cleaned by running 10 ml of water, followed by 10 ml of 70% IPA, with a final rinse of 10 ml water. The nozzle and tubing were air purged with a 10 ml syringe after cleaning.

2 mL of a 3% Alginate solution was combined with 2 ml of ammonium sulfate (pH) gradient liposomes in a centrifuge tube.

The tube was vortexed until the solution was well mixed.

The liposome-Alginate solution was drawn up into a 10 ml syringe.

The volume of the Liposome-Alginate Solution in syringe was 35 mL.

The syringe was placed into the syringe pump. The tubing was attached from the nozzle to the syringe outlet.

3 ml of DI water was drawn up in a separate syringe and placed on the side until needed.

The BAM Collection bowl was filled with 20 g/L CaCL2 solution until the liquid level was at 1 cm.

The nozzle was turned on and set to 5.0 W. The collection dish was stirred at setting 2 with a 1 inch stir bar.

The syringe pump was set to 0.5 mL/min.

When the syringe has been emptied, the syringe was replaced with the syringe earlier set aside. The pump was set to 0.5 mL/min.

After the syringe was emptied, the BAM was incubated for 10 minutes at setting 2. 137 mL of CaCl2 and BAM solution were collected post incubation.

The contents of the dish were collected into 50 ml centrifuge tubes. The tubes were labeled with the notebook number, date, and description.

The tubes were centrifuged at 2500 RPM for 10 minutes. The supernatant in each tube was drawn up and replaced with 20 ml DPBS.

The tubes were resuspended and centrifuged at 2500 RPM for 10 minutes. The supernatant in each tube was drawn up and replaced with 20 ml DPBS.

The tubes were stored at 2-8° C.

### Example 2

Potassium iodide liposomes were manufactured for evaluation in determining radiopacity (FIG. 3C, PTN13-008).

Materials used included potassium iodide, distearoylphosphatidylcholine (DSPC), cholesterol, ammonium sulfate, ethanol, hydrochloric acid, pH paper, and Dulbecco's Phosphate-Buffered Saline (DPBS).

A 480 mg/mL I+ solution of potassium iodide was prepared by dispensing 156.9 g of potassium iodide into a beaker. The solution was diluted with 250 g of deionized (DI) water.

A 300 mM solution of ammonium sulfate was prepared by dispensing 29.6 g of ammonium sulfate into a beaker and dilute with 733.2 g of DI water.

The pH of potassium iodide was adjusted to 5.5 by dispensing 0.625 mL of a 0.12 M hydrochloric acid solution into the potassium iodide solution.

175 mL of the potassium iodide solution was dispensed into a separate 250 ml beaker. The beaker was heated to 65-66° C.

13.1 g of DSPC was added to a 1 L flask and 5.1 g of cholesterol was added into the same flask. 40.2 g of ethanol was dispensed into the flask.

The water bath attached to the rotavap was set to 75° C. The flask was attached to the rotavap once this temperature is reached and the flask was rotated for 15 minutes.

The potassium iodide solution was poured into the flask when the solution is at 65-66° C. after dissolving the lipids in the ethanol.

The contents of the flask were homogenized with the homogenizer at a speed of 4 for two minutes.

The water bath was set to 45° C. and the flask was returned to the water bath once the temperature has been reached.

The flask was cooled to 45° C. for 15 minutes.

While the flask is cooling, the microfluidizer chiller was started and set to 20° C. The microfluidizer was prepared by passing 90-100 g of ammonium sulfate solution through the machine. The pressure was set to 22 kpsi. The remaining amount of ammonium sulfate was removed once at least 5 strokes have been performed with the machine.

After cooling the flask, the contents were dispensed into the microfluidizer. The fluidizer was turned on and the output was collected once the contents started to turn yellow-white.

Four passes were performed by returning the collected output into the hopper once the fluid in the hopper starts to go low four times.

The ethanol in the solution was evaporated by setting the water bath to 28° C. and the vacuum to 28 torr. The solution was rotated in the rotavap at these conditions for 1 hour. The solution was stored at 2-8° C.

### Example 3

Potassium Iodide Liposome BAM were manufactured using the ultrasonic nozzle method and undiluted liposomes (FIG. 3C, PTN13-014).

Materials used included 3% alginic acid; 20 g/L calcium chloride, DPBS, and liposomes.

The nozzle set up was cleaned by running 10 mL of IPA followed by 10 mL of water through the tubing and nozzle and purged with 10 mL of air after cleaning. The waste was collected into a properly named and labeled receptacle.

2 mL of 3% Alginic Acid solution was combined with 2 mL of potassium iodide liposomes into a centrifuge tube. The tube was vortexed until solution is homogenous.

The solution was drawn up into a 10 ml syringe and the volume of Alginate-Liposome Solution was 3.6 mL.

The syringe was placed in the syringe pump, the rubber tubing was attached from the nozzle to the syringe.

4 mL of DI water was drawn up with a separate syringe, and placed on the side until needed.

135 mL of 20% Calcium Chloride Solution was dispensed into a shallow collection bowl and a 2 inch stir bar was placed in the bowl.

The collection bowl was placed below the nozzle with a stir plate underneath with a 1-2 cm gap between the nozzle tip and the surface of the solution.

The nozzle's generator was generated and set to 5.0 W.

The stirring plate was set to 1 so that the solution is slightly agitated by the stir bar.

The syringe pump was activated at 0.5 mL/min.

After the entire contents of the syringe have been dispensed, the syringe pump was stopped and replaced with the syringe set to the side. The syringe pump was turned back on at 0.5 mL/min.

The nozzle was turned off after the syringe was dispensed.

The spheres were incubated in the solution for 10 minutes.

The spheres were dispensed into three 50 mL centrifuge tubes and the total amount collected was recorded. The volume of Alginate-Calcium Chloride Solution was 137 mL. The tubes were centrifuged for 5 minutes at 4000 RPM.

The supernatant was replaced with equivalent amounts of DPBS. The tubes were resuspended.

The tubes were centrifuged again for 5 minutes at 4000 RPM.

The supernatant was removed and replaced with 10 mL of DPBS per tube.

The contents of the tubes were resuspended and transferred into a single tube. The volume of BAM in the tube was 33 mL.

The tube was labeled with the lab notebook number, description, and current date and stored at 2-8° C.

### Example 4

Potassium iodide liposome BAM were manufactured using the ultrasonic nozzle method and undiluted liposomes at a 2:1 ratio (FIG. 3C, PTN13-015).

Materials used included 3% alginic acid, 20 g/L calcium chloride, DPBS, and liposomes.

The nozzle set up was cleaned by running 7_0 mL of IPA followed by 10 ml of water through the tubing and nozzle and purged with 10 mL of air after cleaning. The waste was collected into a properly named and labeled receptacle.

1.2 mL of 3% Alginic Acid solution was combined with 2.8 mL of potassium iodide liposomes into a centrifuge tube. The tube was vortexed until solution is homogenous.

The solution was drawn up into a 10 ml syringe and the volume of the Alginate-Liposome Solution was 3.6 mL.

The syringe was placed in the syringe pump and the rubber tubing was attached from the nozzle to the syringe.

4 mL of DI water was drawn up with a separate syringe and placed on the side until use is needed.

135 mL of 20% Calcium Chloride Solution was dispensed into a shallow collection bowl and a 2 inch stir bar was placed in the bowl.

The collection bowl was placed below the nozzle with a stir plate underneath, with a 1-2 cm gap between the nozzle tip and the surface of the solution.

The nozzle's generator was activated and set to 5.0 W.

The stirring plate was set to 1 so that the solution is slightly agitated by the stir bar.

The syringe pump was activated at 0.5 mL/min.

After the entire contents of the syringe were dispensed, the syringe pump was stopped and the syringe was replaced with the syringe set aside above. The syringe pump was turned back on at 0.5 mL/min.

The nozzle was turned off after the syringe has been dispensed.

The spheres were incubated in solution for 10 minutes.

The spheres were dispensed into three 50 mL centrifuge tubes and the total volume of Alginate-Calcium Chloride Solution was 138 mL.

Thee tubes were centrifuged for 5 minutes at 4000 RPM.

The supernatant was replaced with equivalent amounts of DPBS and resuspended.

The tubes were centrifuged for 5 minutes at 4000 RPM.

The supernatant was removed and replaced with 10 mL of DPBS per tube.

The tubes were resuspended and the contents of the tubes transferred into a single tube.

The total volume of BAM was 37 mL.

The tube was labeled with the lab notebook number, description, and current date and stored at 2-8° C.

### Example 5

The Mastersizer is a laser diffraction particle size analyzer. It can measure particle sizes 0.1 μm-3 mm. Measurements of particle size were made using the Mastersizer. FIG. 3A, FIG. 3B, FIG. 3C, and FIG. 3D depict particle size data comparing 188RNL BAM Intermediates (PTN12-066), KI Liposomes (PTN13-008), BAM KI Liposomes 1:1 (PTN13-014), and BAM KI Liposomes 2:1 (PTN13-015).

### Example 6

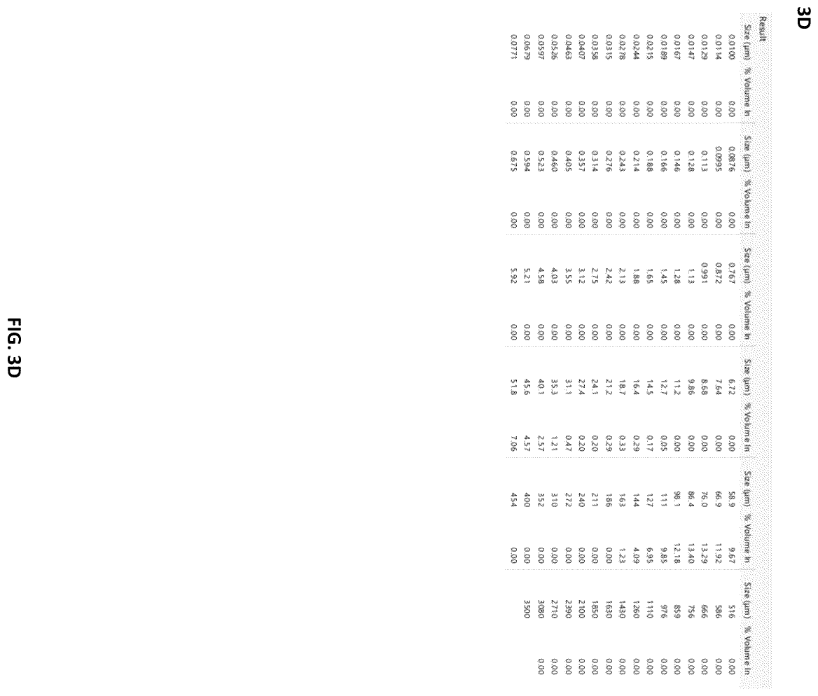

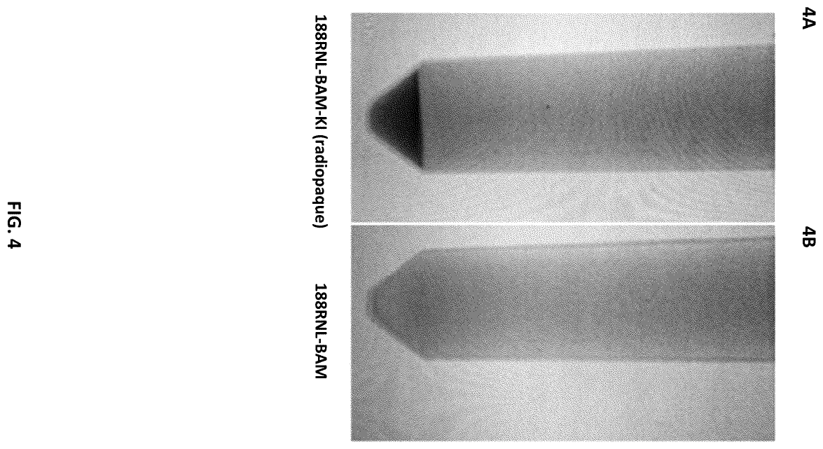

X ray fluoroscopy images with settings of 43 kV, 3.7 mA, 84.55 mGy, for 4 minutes of the radiopaque prototype (KI) to the non-radiopaque prototype (RNL-BAM-KI and RNL-BAM). FIG. 4A and FIG. 4B.

Although the technology herein has been described with reference to embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

### References

1. Jen A C, Wake M C, Mikos A G. Review: hydrogels for cell immobilization. Biotechnol Bioeng. 1996;50(4):357-64.
2. Orive G, et al. Biocompatibility of microcapsules for cell immobilization elaborated with different type of alginates. Biomaterials. 2002;23(18):3825-31.
3. Fischer F G, Dorfel H. Polyuronic acids in brown algae. Hoppe Seylers Z Physiol Chem. 1955;302(4-6):186-203.
4. Smidsrød O, Draget K I. Chemistry and physical properties of alginates. Carbohydr Eur. 1996; 14:6-12.
5. Draget K I, Skjak-Braek G, Smidsrod O. Alginate based new materials. Int J Biol Macromol. 1997;21(1-2):47-55.
6. Haug A, Smidsrod O. The effect of divalent metals on the properties of alginate solutions. II. Comparison of different metal ions. Acta Chem Scand. 1965; 19:341-51.
7. Gupta, A., Clercx, H. J. H., & Toschi, F. (2018). Effect of particle shape on fluid statistics and particle dynamics in turbulent pipe flow. Journal of Fluid Mechanics, 847, 855-879. doi:10.1017/jfm.2018.584.
8. Smith, J. (2019). The role of particle shape on flow dynamics in turbulent pipe flows. International Journal of Fluid Mechanics, 25(3), 189-201. doi: 10.1007/s00348-019-2653-7.
9. Jones, M. (2020). Particle shape effects on turbulent flow dynamics in pipes. Journal of Fluid Mechanics, 892, A36. doi: 10.1017/jfm.2020.129.
10. U.S. Pat. Publ. No. 20220249374.

1. A method of manufacturing a polymeric matrix encapsulating liposomes comprising
adding at least one liposome to a first channel on a microfluidic cassette;
adding at least one cross-linkable polymer to a second channel on the microfluidic cassette;
combining the at least one liposome and at least one crosslinkable polymer to form a solution;
pushing the solution through a third channel on the microfluidic cassette to shear the solution to create individual spheres; and
adding an aqueous solution comprising a cross-linker to a fourth channel on the microfluidic cassette; wherein the cross-linker and cross-linkable polymer form the polymeric matrix encapsulating liposomes.

2. The method of claim 1, wherein the at least one cross-linkable polymer is alginate.

3. The method of claim 2, wherein the alginate is composed of mannuronate and guluronate stereomonomers.

4. The method of claim 3, wherein the alginate is composed of a mannuronate to guluronate ratio of less than or equal to 1.

5. The method of claim 3, wherein the alginate is composed of a mannuronate to guluronate ratio of greater than or equal to 1.5.

6. The method of claim 1, wherein the cross-linker is a divalent cation.

7. The method of claim 1, wherein the cross-linker is a calcium salt.

8. The method of claim 1, wherein the cross-linker is a barium salt.

9. The method of claim 1 further comprising washing the polymeric matrix encapsulating liposomes to remove excess cross-linker.

10. The method of claim 1, wherein the at least one liposome is loaded with at least one active agent before adding the liposome to the first channel on the microcassette.

11. The method of claim 10, wherein the at least one active agent is radioactive.

12. The method of claim 10, wherein the at least one active agent is a beta emitting radioactive nucleotide chelate.

13. The method of claim 12, wherein the beta emitting radioactive nucleotide chelate comprises Rhe188.

14. The method of claim 12, wherein the beta emitting radioactive nucleotide chelate comprises BMEDA.

15. The method of claim 1 wherein the diameter of the first channel, the second channel, the third channel, and the fourth channel is 20-120 μm respectively.

16. The method of claim 1 further comprising an aspect ratio of the microspheres of 0.7-1.3.

17. The method of claim 1 further comprising a difference in flow rate between a dispersed phase and a continuous phase of 0-100 μL/min.

18. The method of claim 1 further comprising a stream temperature of 5° C.-55° C.

19. The method of claim 1 further comprising a stream pressure of 0-1500 mbar.

20. A composition manufactured by the method of claim 1 comprising
at least one liposome;
at least one active agent encapsulated in an intraliposomal aqueous compartment of the liposome;
a cross-linked polymeric matrix, wherein the polymeric matrix is water-insoluble and water-absorbed;
wherein the at least one liposome has a diameter between about 1 nm-200 nm; and
wherein the at least one liposome is embedded in the cross-linked polymeric matrix.

21. The composition of claim 20, wherein the cross-linked polymeric matrix comprises at least one active agent in free form.

22. The composition of claim 20, further comprising an aqueous medium surrounding the cross-linked polymeric matrix embedded with the at least one liposome.

23. The composition of claim 22, wherein the aqueous medium comprises at least one active agent in free form.

24. The composition of claim 20, wherein the at least one active agent is radioactive.

25. The composition of claim 24, wherein the at least one radioactive agent is a beta emitting nucleotide chelate.

26. The composition of claim 20, wherein the cross-linked polymeric matrix comprises alginic acid.

27. The composition of claim 20, wherein the at least one liposome comprises at least one ionic salt inside the intraliposomal aqueous compartment to create an ion gradient.

28. The composition of claim 20, wherein the at least one liposome comprises at least one buffering agent inside the intraliposomal aqueous compartment to create a pH gradient.

29. The composition of claim 20, wherein the at least one liposome comprises at least one radio-opaque material.

30. The composition of claim 20, wherein the cross-linked polymeric matrix has a diameter of about 10-90 μm.

31. The composition of claim 20, wherein the cross-linked polymer is alginate.

32. A method of using the polymeric matrix encapsulating liposomes manufactured using the method of claim 1, wherein the active agent loaded in the polymeric matrix actively destroys tumor cells.

33. A method of using the polymeric matrix encapsulating liposomes manufactured using the method of claim 1 to treat at least one of the following diseases using radioembolization: hepatocellular carcinoma, colorectal liver metastases, paraganglioma liver metastases, n neuroendocrine liver metastases, gastrointestinal liver metastases, breast liver metastases, melanoma liver metastases, pancreatic liver metastases, cholangiocarcinoma liver metastases, colorectal lung metastases, renal lung metastases, cirrhosis-associated thrombocytopenia, metastatic extrahepatic bile duct cancer, glioblastoma, renal cell carcinoma, prostate cancer, and uterine fibroids.
